(19) United States
(12) Patent Application Publication
(10) Pub. No.: US 2026/0182972 A1
(43) Pub. Date: Jul. 2, 2026

(54) DEVICE COMPRISING AN ULTRASONIC TRANSDUCER

(71) Applicant: Withings, ISSY LES MOULINEAUX (FR)

(72) Inventors: Aline CRITON, ISSY LES MOULINEAUX (FR); Matthieu MENANTEAU, ISSY LES MOULINEAUX (FR)

(21) Appl. No.: 19/427,472

(22) Filed: Dec. 19, 2025

(30) Foreign Application Priority Data
Dec. 27, 2024 (FR) FR2415315

(51) Int. Cl. A61B 8/00; A61B 8/06

(52) U.S. Cl. CPC A61B 8/543; A61B 8/06; A61B 8/4227; A61B 8/488; A61B 8/5223

(57) ABSTRACT

A device configured to be positioned on a limb of a user, includes a physiological sensor configured to generate a cardiac signal characteristic of a user's cardiac activity, an ultrasound transducer configured to generate a Doppler signal characteristic of blood flow in the limb, a control unit configured to: receive a cardiac signal from the physiological sensor, analyze the received cardiac signal, and in response to detection of a suspected cardiac arrhythmia in the cardiac signal analysis, activate the ultrasound transducer to generate a Doppler signal.

### CROSS-REFERENCE TO RELATED APPLICATIONS

This application claims priority to French Patent Application No. FR2415315, filed Dec. 27, 2024, the entire content of which is incorporated herein by reference in its entirety.

### FIELD

The present invention relates to the field of measuring devices comprising an ultrasonic transducer, in particular wearable devices.

### BACKGROUND

Cryptogenic stroke is a cerebral infarction whose cause remains unknown after thorough medical evaluation. The immediate strategy in the event of an acute stroke should be based on the search for an indication for medical and/or mechanical cerebral revascularization. After this acute phase, an exhaustive etiological work-up should be carried out to identify the cause of the stroke, if possible, and propose appropriate secondary prevention. With this rather broad definition, it is estimated that 25% of ischemic strokes (a type of stroke that occurs when blood flow to part of the brain is blocked, usually by a clot) can be classified as cryptogenic (Hart RG, Diener HC, Coutts SB, Easton JD, Granger CB, O'Donnell MJ, et al. Embolic strokes of undetermined source: the case for a new clinical construct. Lancet Neurol 2014;13:429-38). North American epidemiological data indicate that approximately 20% of strokes are stroke recurrences (Virani SS, Alonso A, Aparicio HJ, Benjamin EJ, Bittencourt MS, Callaway CW, et al.; American Heart Association Council on Epidemiology and Prevention Statistics Committee and Stroke Statistics Subcommittee. Heart Disease and Stroke Statistics-2021 Update: A Report From the American Heart Association. Circulation. 2021;143:e254-e743).

Atrial fibrillation (or “AF”) is a major known cause of cardioembolic stroke. Studies have shown that transient AF transitions are frequent in a population at high risk of stroke or having had a transient ischemic attack or stroke, but with no documented history of AF (Daoud EG, Glotzer TV, Wyse DG, Ezekowitz MD, Hilker C, Koehler J, et al. Temporal relationship of atrial tachyarrhythmias, cerebrovascular events, and systemic emboli based on stored device data: a subgroup analysis of TRENDS. Heart Rhythm 2011;8:1416-23, Brambatti M, Connolly SJ, Gold MR, Morillo CA, Capucci A, Muto C, et al. Temporal relationship between subclinical atrial fibrillation and embolic events. Circulation 2014;129:2094-9). Subclinical AF increases the risk of ischemic stroke or systemic embolism by 2.5 times. AF may be responsible for up to a third of ischemic strokes in certain populations (Ntaios G, Papavasileiou V, Milionis H, Makaritsis K, Vemmou A, Koroboki E, et al. Embolic Strokes of Undetermined Source in the Athens Stroke Registry: An Outcome Analysis. Stroke. 2015;46:2087-93).

If in-hospital rhythm monitoring does not provide a diagnostic answer, an ECG holter (a wearable device that records the heart's electrical activity continuously over a prolonged period, usually 24 to 48 hours) is recommended. For logistical reasons, ECG recordings made after hospitalization rarely exceed 48 hours. Indeed, very few long-term ECG holters are available.

A meta-analysis of some thirty prospective studies showed that the prevalence of AF was 5% with recordings lasting less than 72 hours, compared with 15% with recordings lasting more than 7 days (Dussault C, Toeg H, Nataan M, Wang ZJ, Roux JF, Secemsky E. Electrocardiographic monitoring for detecting atrial fibrillation after ischemic stroke or transient ischemic attack: systematic review and meta-analysis. Circ Arrhythm Electrophysio. 2016;8:263-9.). Use of an implantable cardiac monitor (ICM) improves to a 30% AF detection rate over three years compared with a 3% rate in the control group (Sanna T, Diener HC, Passman RS, Di Lazzaro V, Bernstein RA, Morillo CA, et al. Cryptogenic stroke and underlying atrial fibrillation. N Engl J Med 2014;26:370:2478-86.). In France, an indication for MCI implantation is recommended for the etiological diagnosis of ischemic strokes, without a cardioembolic source or coagulation disorder having been demonstrated.

These ischemic strokes are most often due to micro-emboli generated during episodes of fibrillation in patients suffering from AF. These micro-emboli can be detected using transcranial Doppler ultrasound. In this technique, a constant-frequency pulsed ultrasound signal scans a volume of blood in the middle cerebral artery, and the micro-emboli moving through its sample volume produce a transient reflection offset from the Doppler effect by a very high amplitude, known as a “high intensity transient signal” (HITS).

Transcranial Doppler ultrasound has a number of material and organizational constraints. In particular, in order to perform these Doppler imaging data, access to ultrasound scanners is needed, which are not devices that can be easily integrated into a patient's daily routine, enabling easy long-term monitoring of patients at risk of AF. This type of imaging examination can only be carried out by a radiologist or a sonographer specialized in transcranial Doppler imaging. These examinations cannot be carried out on an ongoing basis or when an AF episode is present, partly because AF episodes are often asymptomatic, and partly because it is difficult from an organizational point of view to have access to brain imaging examinations at the time of the AF episode, even if the episode is detected.

There have also been a few attempts to incorporate an ultrasound sensor into a watch, as described in US20240130617A1. However, these watches have a number of technical limitations that make them difficult for patients to use in practice.

### SUMMARY

An aspect of the present description is therefore to propose a device that can detect the presence of these micro-emboli during arrhythmia episodes more easily and more systematically, in order to assess and prevent the risks due to a stroke.

To this end, the present description relates to a device configured to be positioned on a limb of a user, the device comprising: a physiological sensor configured to generate a cardiac signal characteristic of a cardiac activity of the user, an ultrasound transducer configured to generate a Doppler signal characteristic of a blood flow in the limb, a control unit configured to: receive a cardiac signal from the physiological sensor, analyze the received cardiac signal, and in response to a detection of a suspected cardiac arrhythmia in the analysis of the cardiac signal, activate the ultrasonic transducer to generate a Doppler signal.

The device according to the present description enables non-invasive and accurate detection of micro-emboli in users with atrial fibrillation. By integrating an ultrasound transducer into a device, such as for example a connected watch, a blood pressure cuff or a scale, it is possible to detect the presence and quantity of micro-emboli in the blood following the detection of an arrhythmia, thus offering an opportunity to prevent strokes in users suffering from AF. In particular, the detection of a micro-embolus can trigger an alert, enabling rapid medical intervention, which is crucial for preventing damage caused by stroke.

In particular, activating the ultrasound transducer temporarily (typically a few minutes) and acquiring a Doppler signal only when an arrhythmia is detected enables optimized use of the device's battery, making long-term, continuous monitoring possible for the user.

Finally, as the device is non-invasive and wearable, it also enhances the user's comfort and quality of life, which in turn increases the user's willingness to use the device on a regular basis.

In an embodiment, a suspected cardiac arrhythmia is a probability of the presence of an arrhythmia above a predetermined threshold, for example a 50% chance.

In an embodiment, the ultrasound transducer is a Doppler probe.

In an embodiment, the arrhythmia is atrial fibrillation.

In an embodiment, the optical sensor is configured to perform a continuous cardiac signal measurement and the control unit is configured to analyze the continuous cardiac signal.

In an embodiment, the cardiac signal is analyzed using a sliding time window.

In an embodiment, the physiological sensor is an optical sensor.

In an embodiment, the physiological sensor and/or an ECG sensor and/or an oscillometric sensor, a ballistograph or ballistocardiograph, BCG, and/or an impedancemetric sensor, IPG.

In an embodiment, the device is a wearable device.

In an embodiment, the optical sensor comprises at least one light source and at least one light receiver.

In an embodiment, the cardiac signal is a photo-plethysmographic signal.

In an embodiment, the optical sensor is configured to measure the cardiac signal periodically, in particular at a frequency of between one measurement every minute and an measurement every 3 minutes, or 5 minutes, or 10 minutes.

In an embodiment, the cardiac signal is an oscillometric signal.

In an embodiment, the cardiac signal is a BCG signal.

In an embodiment, the cardiac signal is an IPG signal.

In an embodiment, the oscillometric sensor is activated during a blood pressure measurement.

In an embodiment, the ECG sensor comprises at least two ECG electrodes made of conductive material.

In an embodiment, the control unit is electrically connected to the two ECG electrodes, and configured to perform an electrocardiogram and detect an arrhythmia in the performed electrocardiogram.

In an embodiment, the ECG module implements a deep learning algorithm.

In an embodiment, the Doppler signal is generated concomitantly with the cardiac signal or less than 10 seconds or even 5 seconds after the cardiac signal.

In an embodiment, the ultrasonic transducer is configured to initiate an ultrasonic measurement in the direction of the limb.

In an embodiment, the ultrasonic transducer emits an ultrasonic signal.

In an embodiment, the ultrasonic transducer comprises at least a ultrasonic sensor, each ultrasonic sensor being configured to emit an ultrasonic signal and receive a signal reflected from the limb.

In an embodiment, the ultrasonic transducer comprises several ultrasonic sensors, in particular to cover a larger area and offer finer resolution through combined analysis of signals from several sensors.

In an embodiment, each ultrasonic sensor comprises a piezoelectric device, in particular made of ceramic.

In an embodiment, the emitted signal is a constant-frequency pulsed signal. The sensor alternates between transmission and reception to analyze specific zones at a certain depth.

In an embodiment, the frequency of the emitted signal is between 1 and 50 MHz, in particular 8 and 15 MHz.

In an embodiment, the ultrasonic transducer is configured to scan the user's limb to locate a user's artery, in particular by analyzing blood velocity, as arteries generally have a faster pulsatile flow than veins.

In an embodiment, when the device is a watch, the ultrasonic transducer is configured to detect the radial artery.

In an embodiment, the ultrasonic transducer is configured to collect Doppler signals of arterial blood flows and micro-emboli circulating in the arteries.

In one embodiment, the control unit is configured to receive the Doppler signal and to analyze the Doppler signal based on the frequency difference between the emitted signal and the reflected signal, typically between 20 Hz and 50 kHz.

In one embodiment, the control unit is further configured to deduce from the Doppler signal the velocity of red blood cells and micro-emboli in the blood circulating in the arteries.

In an embodiment, the ultrasonic transducer is arranged in a wristband of the device.

In an embodiment, the control unit is configured to receive the Doppler signal and configured to detect at least one micro-embolus in the artery by analysis of the Doppler signal.

In an embodiment, the Doppler signal is sent by the device to the external terminal or remote server for analysis.

In an embodiment, the control unit is configured to detect the passage of a micro-embolus by detecting an amplitude variation in the Doppler signal.

In one embodiment, the control unit is configured to detect an intensity peak (called HITS, for “high intensity transient signal”) in the Doppler signal, particularly in relation to the reference amplitude of the surrounding blood flow, characteristic of the passage of a micro-embolus.

In an embodiment, the control unit is configured to emit an alert signal in the event of a detection of a micro-emboli flow rate above a predetermined alert threshold, for example an alert message to the user and/or a third party.

In an embodiment, the alert threshold is between 10 and 100 micro-emboli per minute detected.

In an embodiment, the limb is a wrist or arm of the user.

In an embodiment, the device is a watch.

In an embodiment, the device is a blood pressure monitor.

In an embodiment, the device is a scale.

In an embodiment, the device comprises a rechargeable battery configured to provide power to the device.

An aspect of the invention also relates to an activation method implemented by a control unit of a device as described above, comprising the steps of:
activating the physiological sensor and initiating the generation of a cardiac signal ;
receiving and analyzing the cardiac signal;
in response to a detection of a cardiac arrhythmia in the cardiac signal analysis, activating the ultrasonic transducer.

In an embodiment, the method further comprises controlling a generation of a Doppler signal by the ultrasonic transducer.

In an embodiment, the method further comprises receiving and analyzing the Doppler signal by the control unit.

In an embodiment, the method comprises transmitting the Doppler signal to an external terminal or remote server for analysis.

In an embodiment, the method further comprises, in response to a detection of a micro-emboli, issuing an alert signal.

### BRIEF DESCRIPTION OF THE DRAWINGS

Further features, details and benefits will become apparent from the detailed description below, and from an analysis of the appended drawings, in which:

FIG. 1 shows a schematic representation of a device according to the present description in contact with a user's limb.

FIG. 2 shows two ECG signals (voltage on the ordinate and time on the abscissa), with sinus rhythm (a) and arrhythmia (b).

FIG. 3 shows a Doppler ultrasound with an intensity peak characteristic of the passage of a micro-embolus.

FIG. 4 shows a schematic representation of the device and its environment.

FIG. 5 shows a top view of the device in the form of a watch.

FIG. 6 shows a bottom view of the watch shown in FIG. 5.

FIG. 7 shows a bottom view of the watch shown in FIG. 5, in another embodiment.

FIG. 8 shows a cross-sectional view of the watch shown in FIG. 5.

FIG. 9 shows a perspective view of the device in the form of a blood pressure monitor.

FIG. 10 shows a perspective view of the device in the form of a scale.

FIG. 11 shows a diagram representing a method of activating an ultrasonic transducer according to the description.

### DETAILED DESCRIPTION

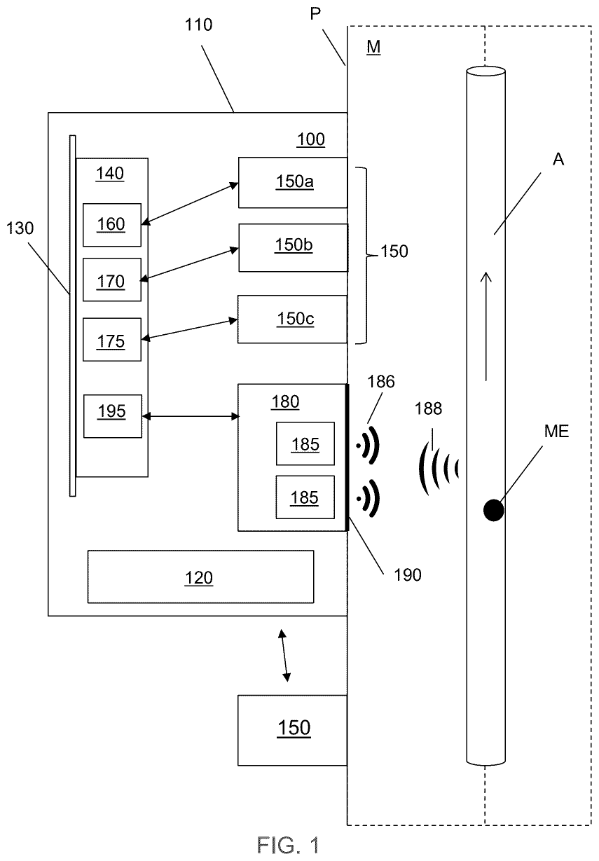

A device 100 is shown in FIG. 1.

In particular, the device is a wearable device. By “wearable”, we mean a device that can be positioned in contact with a user and easily transported by that user. In particular, the device is configured to be positioned on the skin P of a user's limb M, and in particular to be positioned there continuously. In the remainder of the description, “continuously” is taken to mean without interruption over a long period of time, for example at least one hour, in particular at least one full day. However, the device 100 can be removed occasionally, for example for recharging. As will be described in more detail below, the limb M is, for example, a wrist or an arm. However, the description is not limited to these limbs and applies to any limb of the user.

Device 100 comprises an housing 110. The housing 110 forms an enclosure which defines an internal volume suitable for accommodating various components, such as electronic components, as will be explained in more detail below. The housing 110 protects the internal volume in particular from dust, water, humidity and shock. The housing 110 can have a maximum transverse dimension of less than 30 cm, in particular less than 10 cm.

The device 100 may have a mass of less than 1 kg, in particular less than 500 g, in particular less than 250 g and in particular less than 100 g.

Device 100 may comprise a battery 120, located in the internal volume defined by housing 110 or in the wristband. The battery 120 is configured to supply energy to the device 100, and in particular to the electronic components of the device 100. Battery 120 is, for example, a rechargeable battery.

### Control Unit

Device 100 may comprise at least one printed circuit board (PCB) 130. Each PCB is a thin, rigid card containing a printed circuit. The printed circuit board(s) 130 comprises a control unit 140, illustrated schematically in FIG. 1. Other components may also be mounted on the printed circuit board(s) 130.

Control unit 140 is used to control the on-board electronics of device 100. The control unit 140 may, for example, include or partially include a PPG module, an ECG module, an oscillometric module and a Doppler module as will be explained below.

### Physiological Sensor

The device 100 comprises a physiological sensor 150 configured to generate a cardiac signal characteristic of a user's cardiac activity. A signal characteristic of the user's cardiac activity is a physiological measurement that reflects the electrical and/or mechanical activity of the heart, in particular for monitoring or analyzing the functioning of the cardiovascular system. A signal characteristic of the user's cardiac activity is, for example, an electrocardiogram (ECG), a photoplethysmogram (PPG), a phonocardiogram (PCG), a cardiographic impedance measurement (ICG), an oscillometric signal, an impedancemetry signal (IPG), a ballistocardiogram signal (BCG).

The physiological sensor 150 typically generates a continuous cardiac signal. This enables uninterrupted monitoring, including at night when the device 100 is worn at night.

In an embodiment, the physiological sensor 150 is located within the internal volume defined by the housing 110. Alternatively, the physiological sensor 150 can be positioned in a wristband enabling the housing 110 to be attached to the limb M. Alternatively, the physiological sensor 150 can be positioned outside the internal volume defined by the housing 110 and connected to the control unit 140 by an electrical wire or wirelessly, for example via Bluetooth.

### Optical Sensor

In an embodiment, the physiological sensor 150 is an optical sensor 150a. The optical sensor is generally a PPG (photoplethysmography) sensor, comprising at least one light source, for example a LED (light-emitting diode), for emitting a light signal and at least one light receiver, for example a photodiode, for receiving the light signal.

The optical sensor 150a is connected to a PPG module 160, which can be mounted on the printed circuit board 130. The PPG module 160 is configured to generate emission instructions for the LEDs and to retrieve electrical signals from the photodiodes.

In an embodiment, the optical sensor 150a, via the PPG module 160, is configured to perform a continuous cardiac signal measurement. To this end, the light source emits a continuous light signal. The optical sensor is then configured to monitor the user's cardiac activity in real time.

Alternatively, the optical sensor 150a is configured to measure the cardiac signal periodically, in particular at a frequency of between one measurement every minute and one measurement every 2 minutes, or even every 5 minutes, or even every 10 minutes.

### ECG Sensor

Alternatively or additionally, the physiological sensor 150 is an ECG sensor 150b, configured to measure an electrocardiogram (“ECG”) of the user.

The ECG sensor 150b is configured to receive electrical signals generated by the human body. In particular, the ECG sensor 150b comprises a set of electrodes (hereinafter referred to as ECG electrodes) and is connected to an ECG module 170, which can be mounted on the printed circuit board 130 and to which the ECG electrodes are electrically connected. By electrode we mean a conductive part capable of receiving an electric current or voltage. The part may be made of a conductive material or have a conductive coating. By “conductive” we mean “electrically conductive”.

In an embodiment, device 100 may comprise just two ECG electrodes. Alternatively, the device 100 may comprise a third ECG electrode.

ECG module 170 is configured to retrieve electrical signals from the human body and to generate, after processing, an electrocardiogram signal or data representative of electrocardiogram information. To this end, the ECG module 170 can implement a deep learning algorithm for processing and analyzing the ECG signal.

The ECG sensor 150b is more constrained in terms of positioning on the body, since a loop is required to generate an ECG signal.

In an embodiment, the ECG electrodes are arranged on the housing 110.

Alternatively, at least one ECG electrode is spaced apart from the housing 110 and wired or wirelessly connected to the control unit 140.

To measure a continuous ECG, the ECG sensor 150b can be a sensor attached to the torso.

### Oscillometric Sensor

Alternatively or additionally, the physiological sensor 150 is an oscillometric sensor 150c, configured to perform an oscillometric measurement of the user.

In particular, an oscillometric sensor is used to measure a user's blood pressure non-invasively, by detecting pressure variations in an inflatable cuff placed around the arm or wrist.

The optical sensor 150c is connected to an oscillometric module 175, which can be mounted on the printed circuit board 130. The oscillometric module 175 is configured to generate oscillometric measurement instructions.

In particular, the oscillometric module 175 is configured to detect pressure oscillations caused by the heart beating in the artery.

### Ultrasonic Transducer

Device 100 comprises an ultrasonic transducer 180 configured to generate a Doppler signal characteristic of blood flow in limb M. In particular, ultrasonic transducer 180 is configured to initiate an ultrasonic measurement in the direction of limb M. The ultrasonic transducer may also be referred to as a Doppler probe.

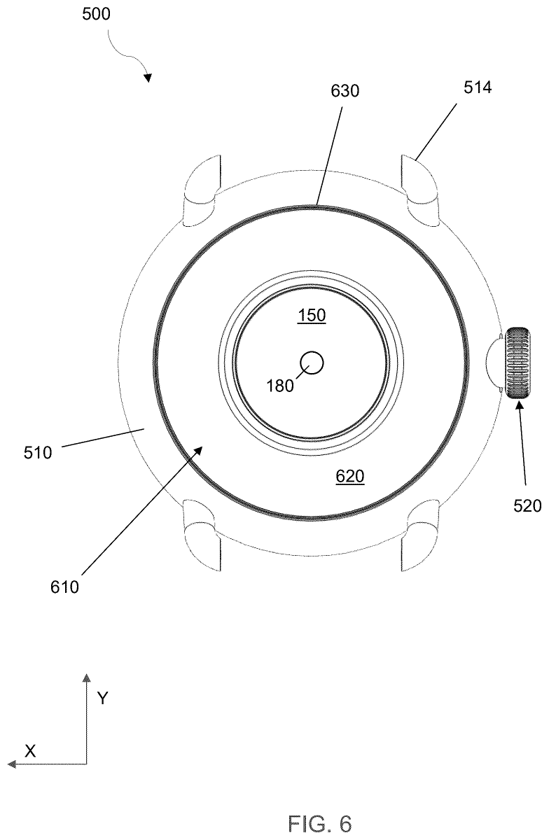

In FIG. 1, the ultrasonic transducer 180 is arranged in the internal volume defined by the housing 110. Alternatively, however, the ultrasonic transducer 180 can be positioned in a wristband enabling the housing 110 to be attached to the limb M (as shown, for example, in US20240130617A1, FIG. 6 and paragraph 183).

To this end, ultrasonic transducer 180 comprises at least one ultrasonic sensor 185. Each ultrasonic sensor 185 is configured to emit an ultrasonic signal 186 and receive a reflected signal 188 from the limb M and in particular from the limb's cardiovascular system, i.e. essentially from one or more arteries present locally in the limb M. In an embodiment, the ultrasonic transducer 180 comprises several ultrasonic transducers 185, in particular to cover a larger area in the limb M and offer finer resolution thanks to the combined analysis of signals from several ultrasonic transducers.

The ultrasonic sensor(s) 185 are chosen from micromachined capacitive ultrasonic sensors (CMUT), micromachined piezoelectric ultrasonic sensors (PMUT) or piezoelectric sensors. In an embodiment, the ultrasonic sensors are of the capacitive micromachined ultrasonic (CMUT) or piezoelectric micromachined ultrasonic (PMUT) type. In an embodiment, the ultrasonic sensors are capacitive micromachined (CMUT).

In an embodiment, the ultrasonic sensor(s) comprises at least one strip comprising at least two elements. In an embodiment, the ultrasonic sensors are isolated elements.

The signal emitted by each ultrasonic sensor 185 may be a pulsed signal at a constant frequency. Each ultrasonic sensor 185 is configured to alternate between emitting a signal and receiving a signal, in particular to analyze specific zones at a certain depth in limb M. Each ultrasonic sensor 185 is configured to emit a sound signal at a frequency of between 1 and 50 MHz, in particular between 8 and 15 MHz.

Alternatively, ultrasonic transducer 180 is configured for continuous Doppler signal transmission and acquisition.

The ultrasonic transducer 180 is configured to generate the Doppler signal based on the frequency difference between the emitted signal and the reflected signal, due to the Doppler effect. The frequency difference is in particular between 20 Hz and 20 kHz.

Ultrasonic transducer 180 is configured to emit a Doppler signal with focused (or “beamformed”) beams or unfocused beams, in particular as a plane wave or spherical wave.

In an embodiment, the ultrasound transducer 180 is configured to scan the user's limb M to locate an artery A of the user. In particular, ultrasonic transducer 180 is configured to modify the direction of emission of ultrasonic signals, for example by moving one or more piezoelectric elements. The location of the artery A is determined in particular by analyzing the speed of the blood in the various vessels detected, arteries generally having a faster pulsatile flow than veins.

To make contact with the skin of the limb, the ultrasound transducer 180 may comprise an interface 190. In an embodiment, interface 190 comprises a flexible material, such as silicone, arranged to contact the skin, to improve acoustic coupling with the skin.

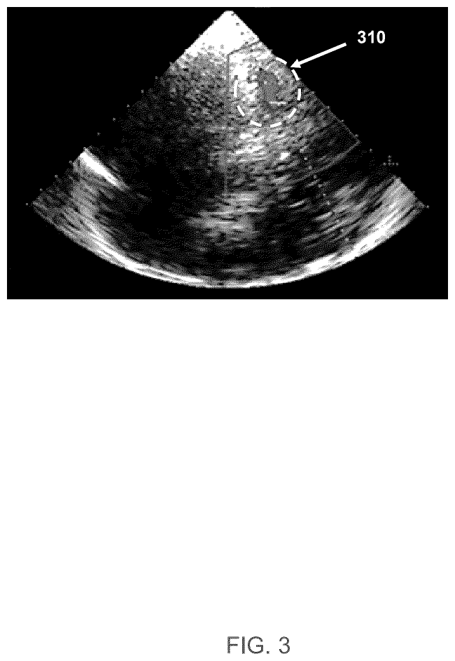

In particular, the ultrasound transducer 180 can be used to generate Doppler images, as shown in FIG. 3 and described in greater detail below.

### Architecture

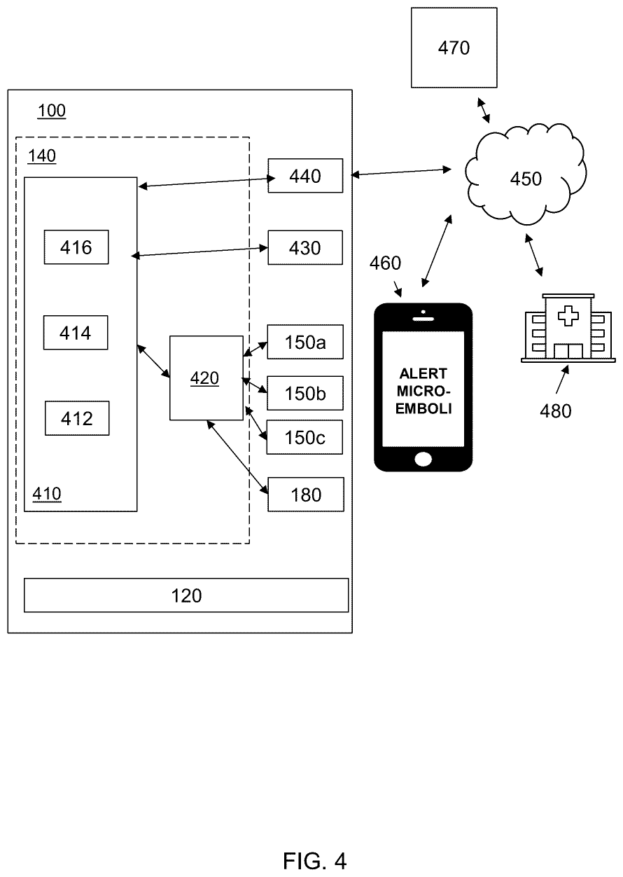

FIG. 4 shows a schematic diagram of the architecture of a device 100 as described and its environment.

The control unit 140 comprises control circuitry 410 including a processor 412, a memory 414 and an I/O interface 416 for communication with other components.

Memory 414 stores programs, instructions or other items that enable the device 100 to be navigated and measurements to be taken (algorithms in particular). In particular, memory 414 can be broken down into volatile, RAM-type memory and non-volatile, flash-type memory (or ROM or SSD).

The control unit 140 comprises the optical module 160, the ECG module 170, the oscillometric module 175 and/or the Doppler module 195. In particular, control unit 140 is configured to control activation and measurement by physiological sensor(s) 150 and/or ultrasound transducer 180.

In particular, the control unit 140 is arranged on a printed circuit board, the so-called main printed circuit board 130. The control unit 140 may consist of several sub-units, arranged on the main printed circuit board 130 and on other printed circuit boards.

Control unit 140 typically comprises an interface module 420 interfacing between physiological sensor(s) 150, ultrasound transducer 180 and I/O interface 416 of control circuitry 410. The interface module 420 includes ADCs, filters, amplifiers, etc.

The device 100 also includes the display 430, which communicates with the I/O interface 416.

The battery 120 is configured to supply the various components with electrical power for the device 100, for example a battery or a rechargeable battery. In particular, battery 120 is configured to power control unit 140, display 430, physiological sensor(s) 150 and ultrasound transducer 180.

### Connectivity

Still referring to FIG. 4, the device 100 includes a wireless communication module 440, such as a Bluetooth® or Bluetooth Low Energy® module or a Wi-Fi module or a cellular module (GSM, 2G, 3G,4G, 5G, Sigfox, etc.), which enables it to communicate, via a communication network 450, bidirectionally with at least one external terminal 460, such as a smartphone. The external terminal 460 can also communicate (bidirectionally) with the remote server 470 for data storage and processing. Alternatively or additionally, the wireless communication module 440 can communicate directly with the remote server 470, for example via the cellular network or via a Wi-Fi network.

Via the communication network 450, the external terminal 460 or the remote server 470 can communicate with a help desk 480.

### Control Unit—Add-Ons

### Cardiac Signal Analysis

The control unit 140 is configured to receive a cardiac signal from the physiological sensor(s) 150. The cardiac signal is in particular an optical signal and/or an ECG signal.

The control unit 140 is configured to analyze the received cardiac signal. In particular, control unit 140 is configured to characterize the received cardiac signal, in particular to detect the suspicion of the presence of a cardiac arrhythmia. Suspicion of presence is a probability of the presence of an arrhythmia in the cardiac signal greater than a predetermined threshold. The predetermined threshold is, for example, equal to a probability of 50%. The arrhythmia detected is, in particular, atrial fibrillation. Analysis of the cardiac signal by the control unit 140 of the device 100 itself saves battery power and enables an analysis to be carried out regardless of the state of connectivity of the device 100 with the external terminal 460 or the remote server 470, and without delays associated with sending the cardiac signal to the external terminal 460 or the remote server 470 (so that the signal can be analyzed there) and receiving the analysis.

In particular, control unit 140 can be configured to implement pre-processing of the received cardiac signal. Pre-processing includes, for example, resampling the signal to a frequency that can be further processed and/or filtering to eliminate noise (movement, artifacts, etc.).

The control unit 140 can be configured to implement processing of the received signal. Processing includes, for example, detecting pulsation peaks and analyzing the interval between these peaks (known as the RR interval, named after the R peak in the ECG QRS complex).

Arrhythmia identification using a cardiac signal, in particular an ECG signal, a PPG signal, an oscillometric signal, a BCG signal or an IPG signal, is known and will not be described in detail. For example, WO2024194199 describes a watch-embedded algorithm for detecting arrhythmias. For example, Pereira (Pereira, T., Tran, N., Gadhoumi, K. et al. Photoplethysmography based atrial fibrillation detection: a review. npj Digit. Med. 3, 3 (2020). https://doi.org/10.1038/s41746-019-0207-9) presents a review of atrial fibrillation detection using PPG signals.

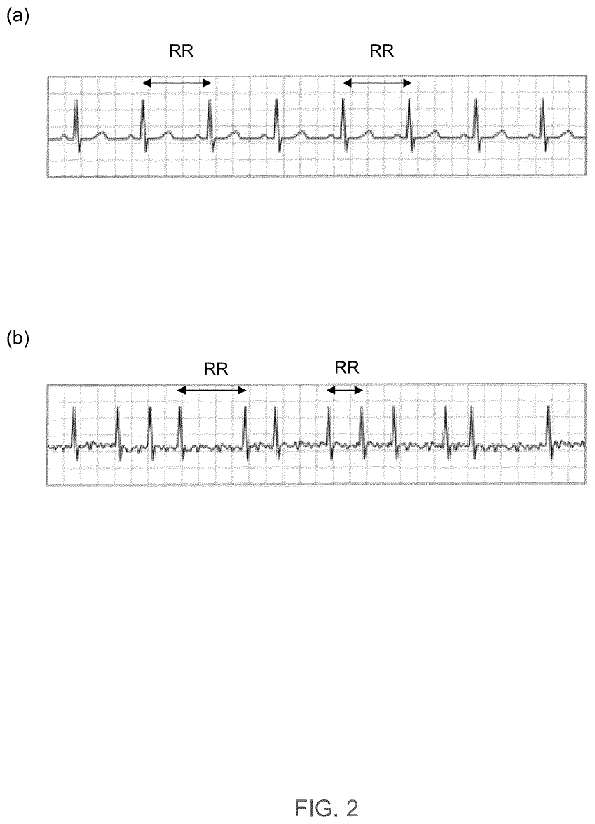

FIG. 2 shows an ECG signal in case (a), where the RR intervals are regular and the cardiac signal is said to be “normal” or in “sinus rhythm”. In case (b), the RR intervals are irregular, characteristic of irregular cardiac activity. The cardiac signal then exhibits an arrhythmia, which may in particular be atrial fibrillation.

The control unit 140 can be configured to implement a classification of the cardiac signal, in particular on the basis of the pre-processed and/or processed signal. The characterization of the cardiac signal can be implemented using a deep learning algorithm, for example of the classifier type. For example, the control unit 140 can execute one or more machine-learning or deep-learning models trained to extract morphological, temporal and spectral features from the cardiac signal (e.g., beat-to-beat interval variability, pulse morphology, peak dispersion, or waveform distortion), to generate a robust characterization of the user's cardiac rhythm. The classifier may comprise, for example, a convolutional neural network (CNN), a recurrent neural network (RNN), a transformer-based model, or a hybrid architecture combining convolutional layers for feature extraction with temporal layers for rhythm-pattern recognition. The model can be trained using supervised learning on large annotated datasets to detect arrhythmic patterns, artifacts, and noise. The classification results can be, for example, the following classes: “poor signal quality”, “atrial fibrillation”, “other arrhythmia”, “sinus rhythm”, “other”. In some embodiments, the classifier outputs both (i) a discrete class label and (ii) a continuous confidence score or arrhythmia-probability value, which may be used by the control unit 140 to determine whether an arrhythmia-trigger threshold has been exceeded and whether to activate the ultrasonic transducer 180.

In an embodiment, control unit 140 can be configured to implement regression. The control unit 140 can be configured to assign a value to the analyzed signal, in particular a value representing the suspicion of the presence of an arrhythmia, in other words the probability of the presence of an arrhythmia.

The control unit 140 is configured to continuously analyze the cardiac signal received. In particular, the analysis can be performed using a sliding time window on the received cardiac signal, for example a sliding window of 30 s. This method enables real-time analysis: the arrhythmia can thus be detected in real time, i.e. instantaneously in relation to its occurrence. Alternatively, the analysis can be carried out using successive signal portions, for example successive 30 s portions. This method is less energy-intensive, but creates a delay of between 0 and 30 s for the analysis. The analysis can also be performed over a longer time interval, for example at least one minute, to detect an arrhythmia in the cardiac signal several times during this time interval to confirm the presence of an arrhythmia.

### Ultrasound Transducer Activation

The control unit 140 is configured to activate the ultrasonic transducer 180 in response to a detection of a cardiac arrhythmia in the cardiac signal analysis. In other words, unless expressly activated for other reasons (e.g. voluntary action by the user), within the scope of the present description, the ultrasonic transducer 180 is activated and performs measurements only when an arrhythmia is detected in the cardiac signal received and analyzed by the control unit 140.

Activation takes place without delay after the detection of an arrhythmia in order to optimize the chances of obtaining a Doppler signal characteristic of the blood flow in the limb concomitantly or very shortly (as little as possible) after the occurrence of the arrhythmia. In particular, “without delay” is to be understood as an activation that occurs automatically and immediately upon satisfaction of the arrhythmia-detection criterion, subject only to inherent and unavoidable processing latencies of the control unit 140. Such latencies typically include microcontroller computation time, data-buffer transfer time, and electronic switching time, and may be on the order of milliseconds up to a few seconds (e.g., less than 1 s, less than 2 s, or less than 5 s). In an embodiment, activation occurs during the same analysis window in which the arrhythmia is detected, or within a predetermined maximum response time configured to be sufficiently short to ensure that the Doppler acquisition overlaps temporally with the arrhythmic cardiac event.

When the ultrasonic transducer 180 is activated, the ultrasonic transducer 180 is configured to initiate a measurement instantaneously or a few seconds after activation, so that the Doppler signal generated by the ultrasonic transducer is concomitant with or generated a few seconds after the cardiac signal comprising an arrhythmia. A few seconds means less than 10 seconds.

The control unit 140 is configured to receive the Doppler signal generated by the ultrasound transducer 180. Control unit 140 is further configured to deduce from the Doppler signal the velocity of red blood cells in the blood circulating in artery A.

In an embodiment, the control unit 140 is further configured to analyze the Doppler signal in order to detect at least one micro-embolus ME in the artery A. In particular, control unit 140 is configured to measure a quantity of micro-emboli ME per unit of time, for example per minute. This on-board analysis enables the device 100 to be autonomous in detecting embolism or the micro-embolus, without the need for the device 100 to communicate with the external terminal 460 or the remote server 470.

Alternatively, the Doppler signal is sent by the device 100 to the external terminal 460 or remote server 470 for analysis, in order to benefit from increased computing capacity compared with the on-board control unit 140.

In particular, analysis to detect the passage of micro-emboli ME may involve detecting an amplitude variation in the Doppler signal. In this case, the analysis involves detecting an intensity peak (HITS, for “high intensity transient signal”) in the Doppler signal, relative to the reference amplitude of the surrounding blood flow, characteristic of the passage of at least one micro-embolus ME. Such a peak 310 is illustrated in FIG. 3, for example, which shows a Doppler ultrasound obtained by an ultrasound transducer 180. The amplitude of the micro-emboli is generally 10 to 20 dB higher than that of the blood flow signals.

Analysis can be performed using advanced techniques such as Fast Fourier Transform (FFT), wavelet transforms and threshold filtering to improve embolic signal detection by isolating high-amplitude transient peaks in the Doppler spectrum and/or the classification of embolic signal signatures, a combination of spectral analysis and signal clustering techniques to identify embolic signatures, custom-developed convolutional neural networks (CNNs) for image-based spectrogram analysis, machine learning models trained to classify micro-emboli Doppler signals, or methods combining two or more of the above.

Doppler signal analysis can be performed with a delay or a time lag, as long as the Doppler signal is acquired as soon as possible after an arrhythmia detection.

In an embodiment, the control unit 140 is configured to leave the ultrasonic transducer 180 active for a predetermined time interval, for example between 10 seconds and 5 minutes, or between 10 seconds and 1 minute. The control unit 140 is configured to deactivate the ultrasonic transducer 180 at the end of this time interval, to save the battery 120. Thus, in an embodiment, the Doppler signal, unlike the cardiac signal, is not received continuously by the control unit 140 but only during limited time intervals, just after a detection of an arrhythmia in the cardiac signal.

Atrial fibrillation (or “Afib”) is a major risk factor for stroke, due to the formation of blood clots in the heart that can migrate to the brain. During Afib, the atria do not contract efficiently. This leads to blood stagnation, which encourages the formation of blood clots. These blood clots can be ejected from the heart when the heart pumps. This ejected clot then becomes a micro-embolus. If a micro-embolus reaches the cerebral arteries, it can obstruct blood flow and cause an ischemic stroke. The control unit 140 can therefore detect such a micro-embolus following the detection of a cardiac arrhythmia event, in particular atrial fibrillation. Continuous or even real-time arrhythmia detection enables monitoring at any time.

### Issuing an Alert

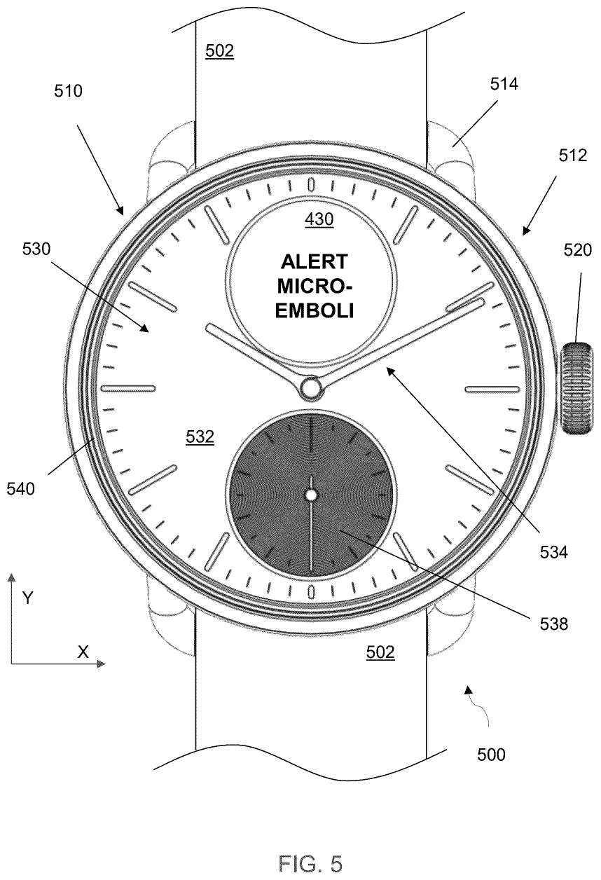

The control unit 140 is configured to emit an alert signal in the event of the detection of a micro-emboli ME flow rate greater than an alert threshold combined with the detection of an arrhythmia. The alert threshold is, for example, between 10 and 100 micro-emboli per minute detected. The alert signal can be an alert message displayed on a display 430 of the device 100, as illustrated in more detail on the device 100 with a watch 500 shape of FIG. 5. Alternatively or additionally, the alert signal is an alert message sent to the user, for example on an external terminal 460 of the user, such as a smartphone, as illustrated in FIG. 4. Alternatively or additionally, the alert signal can be an alert message sent to a third party, such as a relative or medical personnel from a assistance service 480, who is monitoring the user remotely.

Issuing an alert allows the user to react quickly to the risk of stroke, and in particular enables rapid medical intervention, which may be crucial in preventing damage caused by a stroke.

In one embodiment, the control unit 140 is configured to also emit an alert signal upon detection of an arrhythmia, without detection of a micro-embolus ME, in particular to enable medical support for the user.

### Watch

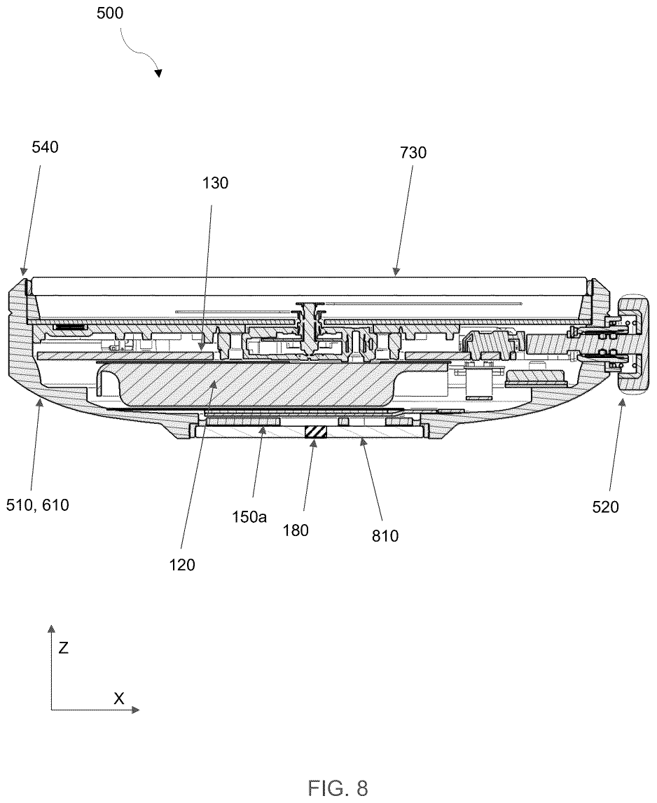

In one embodiment, the wearable device 100 is configured to be placed on a user's wrist. In this embodiment, the device 100 is, for example, a watch 500, as illustrated in FIGS. 5 to 8. The watch 500 may comprise a bracelet 502. The device may also be an activity tracker.

In particular, FIGS. 5 to 8 illustrate an electronic watch 500, of the hybrid type, with a dial, mechanical hands, and possibly a display integrated into the dial. A standard reference frame (XYZ) is shown on these figures.

In a variant not shown, the watch may be a non-hybrid watch without mechanical hands but with a display screen.

The housing 110 of the device 100 may comprise a case 510 and a case back 610, as shown in FIG. 6.

When the device 100 is a watch, the case 110 may also be referred to as a “case”. The case 110 may comprise a side wall 512 which is generally visible when the watch 500 is worn on the wrist. The case 510 may include tabs 514 for attaching a bracelet (not shown in the figures). In particular, the case 510 may comprise two pairs of tabs 514, on either side of the case 510. The case 510 may comprise a plurality of parts.

The watch 500 also comprises a crown 520 projecting through the case 510. As shown in FIGS. 5 to 7, the crown 520 projects orthogonally to the Z axis, along an X axis.

In the illustrated embodiments, the watch 500 comprises a single crown 520. Alternatively, the watch 500 may comprise a plurality of crowns 520. In particular, the crown 520 acts as a user interface between the watch 500 and the user. In particular, the crown 520 can be used by the user to set the time or date, to navigate through the menu, and/or to start recording an activity, etc. The crown 520 can be a push-button and/or a rotating wheel.

The case back 610 is the rear face of the watch 500. The case back 610 is configured to be at least partially in contact with the skin of the user's wrist. In an embodiment, the case back 610 may comprise at least in part the physiological sensor(s) 150, such as an optical sensor 150a or an ECG sensor 150b. For example, the bottom may comprise an annular element 620. In particular, the annular element 620 may surround the optical sensor 150a and the ultrasound transducer 180.

In an embodiment illustrated in FIG. 6, the case 510 and the caseback 610 are two separate parts. In this embodiment, the case 510 and the case back 610 may be separated by a gasket 630.

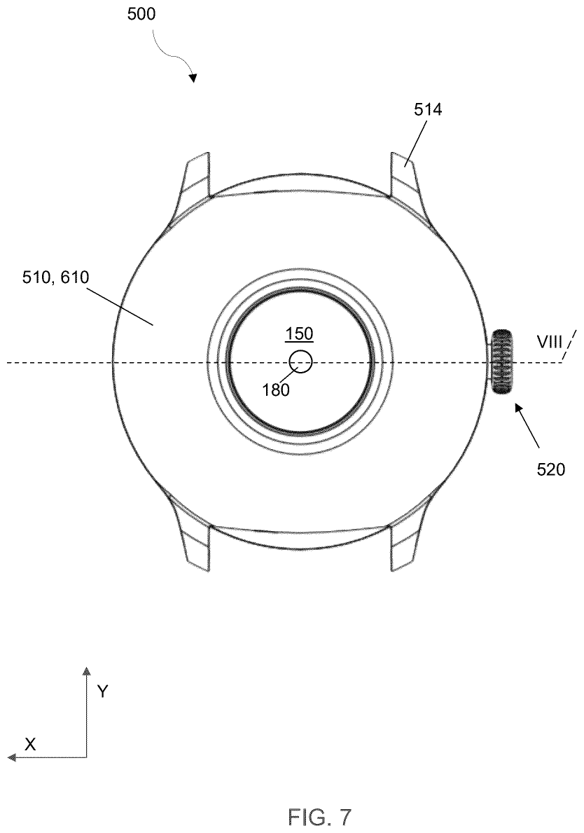

In a variant illustrated in FIG. 7, the case 510 and the case back 610 are integral with each other. In this embodiment, the case 510 and case back 610 constitute a single mechanical part.

The watch 500 may also include a glass 530 (also called “crystal”), typically mounted on the case 510, so that the glass 530 is fixed. The glass 530 may comprise a typically transparent protective glass and may be made of glass, ceramic, plastic or any other transparent material. The contour of the glass 530 is typically circular.

In the case of a hybrid watch, beneath the glass 530, the watch 500 further comprises a dial 532 with physical hands 534. The dial 532 can also accommodate the display 430, here formed by a screen (for example, with an opening in the dial that allows a screen positioned just below the dial to be visible), which occupies, for example, a small space below or inside the dial 532. In particular, the display 430 is configured to show alert messages for the user.

The watch 500 can also include an additional dial 538 to display, for example, the daily number of steps taken by the user or another indicator of the amount of physical activity. The glass 530 protects these parts and allows them to be seen through.

In the case of an Apple Watch™ type “smartwatch”, not shown in the figures, below the glass 530, the watch 500 comprises a digital display that occupies a width close to the width of the watch 500. In an embodiment, the screen can display hands. The glass 530 is then the protective glass for the display.

The watch 500 may also include a bezel 540, mounted on the case 510. The bezel 540 is positioned around the glass 530 (radially external to the glass around the Z direction). In the illustrated embodiments, bezel 540 is fixedly mounted relative to case 510. In an embodiment not shown, the bezel 540 may be rotatably mounted relative to the case 510.

### Optical Sensor

The physiological sensor 150 may be an optical sensor 150a. The optical sensor 150a is located on the case back 610. The optical sensor 150a is typically a PPG (photoplethysmography) sensor, comprising LEDs to emit light and photodiodes to receive the light. The optical sensor 150a can be placed behind a lens 810, such as a glass lens, which interfaces with the skin of the wrist. Document PCT/EP2021/058955, assigned to Withings™, and incorporated by reference, describes in detail an embodiment of the optical sensor, found in particular on the Withings ScanWatch™ and the Withings ScanWatch Light™. Document EP24192447.1, assigned to Withings™, and incorporated by reference, describes in detail another embodiment of the optical sensor, found in particular on the Withings ScanWatch 2™.

### ECG Sensor

Alternatively or additionally, the physiological sensor 150 can be an ECG sensor 150b. The ECG sensor 150b comprises at least two ECG electrodes.

The first ECG electrode, is located on the case back 610 so as to be in contact with the skin of the user's wrist on which he is wearing the watch 500. The first ECG electrode is electrically connected to the ECG module 170. In an embodiment, the case back 610 is the first ECG electrode. In this embodiment, the case back 610 is made of an electrically conductive material, such as metal.

In an embodiment, the first ECG electrode is arranged on the lens 810, for example with a metal coating.

The first ECG electrode may at least partially surround the optical sensor 150b.

The second ECG electrode can be arranged on the bezel 540, so that the user can touch any part of the bezel 540 to perform an ECG. By part of the bezel 540, we mean any part of the bezel surface accessible to the user by touch. In this embodiment, the crown 520 is not an ECG electrode.

In an embodiment, the second ECG electrode is arranged on the crown 520.

In an embodiment, the ECG sensor 150b may comprise a third ECG electrode, for example arranged on the case back 610.

### Oscillometric Sensor

Alternatively or additionally, the physiological sensor 150 can be an oscillometric sensor 150c. The oscillometric sensor 150c comprises, for example, in the form of a bracelet with an inflatable airbag. Such a sensor is described, for example, in documents WO2024140132A1 and US20200345248A1.

### Ultrasonic Transducer

Ultrasonic transducer 180 may comprise a miniaturized ultrasonic sensor. Miniaturized means that the maximum transverse dimension of the ultrasonic transducer is less than 5 mm, in particular less than 2 mm. In particular, the ultrasonic sensor is a piezoelectric transceiver.

In an embodiment, the ultrasonic transducer 180 is arranged on the case back 610 to be in contact with the user's skin when the watch 500 is worn.

In particular, the ultrasonic transducer 180 is positioned in the middle of the case back 610, so as to improve contact between the ultrasonic transducer 180 and the user's wrist and minimize disturbances due to wrist movements.

Alternatively, ultrasonic transducer 180 is arranged in wristband 502.

In particular, ultrasonic transducer 180 is configured to detect the radial artery, which is the main artery of the forearm.

### Blood Pressure Monitor

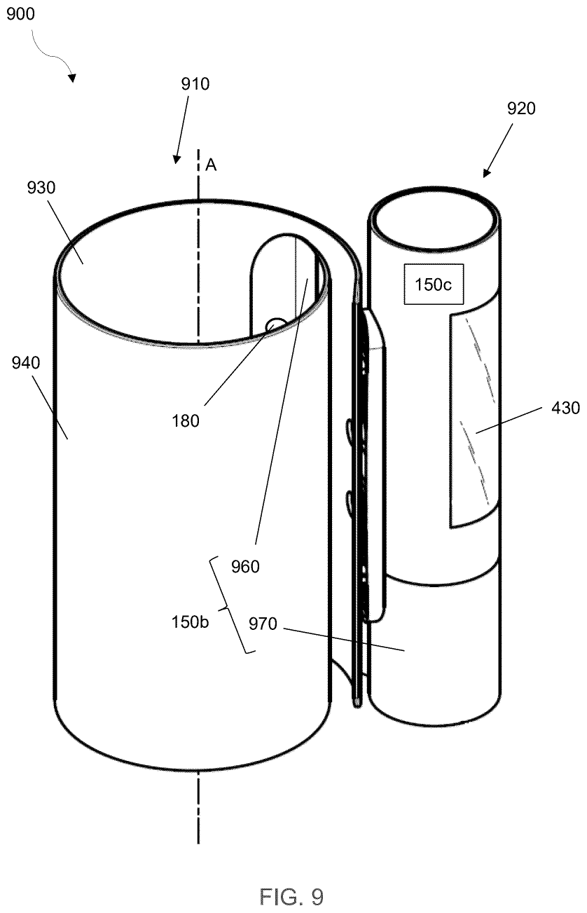

In an embodiment, the wearable device 100 is configured to be placed on a user's arm. In this embodiment, the device is, for example, a blood pressure monitor 900, as illustrated in FIG. 9.

The blood pressure monitor 900 comprises a cuff 910 and a control box 920.

In the illustrated embodiment, the cuff 910 and the control box 920 are mechanically integral. Alternatively, control box 920 may be connected solely to control box 920 by a flexible conduit.

The cuff 910 is configured to be wrapped around a user's arm, in particular around the part of the upper limb between the shoulder and the elbow. Alternatively, the blood pressure monitor 900 can be used elsewhere, on the forearm for example, or on the wrist. Generally speaking, the cuff is configured, in use, to surround a user's upper limb extending along a principal axis A.

Cuff 910 comprises an inflatable pouch, not visible in the figures, disposed between an inner wall 930 configured to be a contact of the user's arm and an outer wall 940.

The cuff 910 is configured to change from an expanded configuration to a rolled-up configuration, as illustrated in FIG. 4. Cuff 910 can include a spring, arranged between inner wall 930 and outer wall 940, constraining cuff 910 in the rolled-up configuration.

In the expanded configuration, the length of cuff 910 along the longitudinal direction, orthogonal to the main axis A, may be between 20 cm and 40 cm. The height along the main axis A can be between 10 cm and 20 cm.

The control box 920 forms the housing 110. The control box 920 can extend substantially along the main axis A.

Control box 920 comprises a pneumatic unit including a pump driven by an electric motor. The pump is configured to inflate the inflatable pocket of the cuff 910.

The control box 920 is configured to control the pneumatic unit and to determine at least the user's blood pressure.

The control unit 920 can include a display 430 configured to display, in particular, a menu for selecting available functions, the results of measurements taken and/or messages intended for the user, in particular warning messages.

The control box 920 is generally cylindrical in shape. The diameter of the control box is, for example, less than 40 mm, which makes it possible to house all the components required for measurement in a compact manner.

The blood pressure monitor 900 also includes a battery 120, housed for example in the control box 920 and configured to supply power to the blood pressure monitor 900, and in particular to the electronic components of the control box 920.

### ECG Sensor

In an embodiment, the physiological sensor 150 is an ECG sensor 150b.

For this purpose, at least two electrodes, in particular three contact electrodes, are provided, all three integrated in the blood pressure monitor 900, without the need for connecting wires and floating electrodes.

The first electrode 960 is arranged on the inner wall 930 of the cuff 910 and has a side facing the user's skin.

The second electrode 970 is arranged around the outer wall of the control box 920, as shown in FIG. 9. The second electrode 970 comprises a conductive material covering at least part of the control box 920.

In an embodiment not shown, a third electrode can also be arranged on the inner wall 930 of the cuff 910, and also has a side facing the user's skin.

### Optical Sensor

Alternatively or additionally, in an embodiment not shown, the physiological sensor 150 is an optical sensor 150a. The optical sensor 150a is arranged on the inner wall 930 of the cuff 910 and faces the user's skin.

### Oscillometric Sensor

Alternatively or additionally, the physiological sensor 150 can be an oscillometric sensor 150c. The oscillometric sensor 150c is connected to the pneumatic unit to measure the user's blood pressure.

### Ultrasonic Transducer

The ultrasonic transducer 180 may comprise at least one miniaturized ultrasonic sensor. As used herein, “miniaturized” refers to a sensor or transducer assembly having a sufficiently small form factor to be integrated into a wearable device without impairing user comfort or continuous skin contact. In particular, miniaturized means that the maximum transverse dimension of the ultrasonic transducer is less than 5 mm, in particular less than 2 mm. In certain embodiments, the thickness of the transducer may also be constrained to less than 1-2 mm to facilitate integration into a watch case, wristband, blood-pressure cuff, or scale platform. The miniaturized construction may employ micromachined structures or compact piezoelectric stacks to achieve both reduced dimensions and adequate acoustic output. The ultrasonic transducer is in particular a piezoelectric transceiver. In such embodiments, the piezoelectric transceiver includes one or more piezoelectric elements configured to operate in both transmission and reception modes, thereby enabling the device to emit ultrasonic pulses and detect corresponding echoes reflected from vascular structures within the limb.

The ultrasonic transducer 180 is arranged on the inner wall 930 of the cuff 910 to be in contact with the user's skin when the blood pressure monitor 900 is worn.

In particular, ultrasonic transducer 180 is configured to detect the brachial artery, which is the main artery of the upper arm.

### Scale

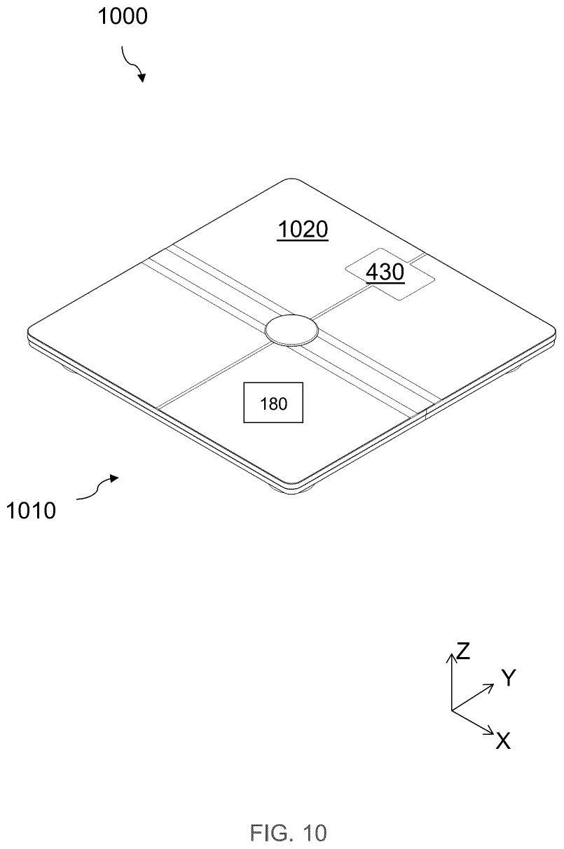

In an embodiment, the device 100 is configured to be in contact with a user's feet. In this embodiment, the device is, for example, a scale, as illustrated in FIG. 10.

The scale 1000 is essentially in the form of a base 1010 on which a user can place his or her feet, for example flat. The user can stand on the base 1010 or sit on a chair.

The scale 1000 may further comprise weight sensors, such as load cells, capable of measuring a user's weight. The weight sensors can also be used to perform a BCG (ballistocardiogram), i.e. a measurement of weight variation under the effect of blood ejection from the heart.

The scale 1000 may take the form of an impedancemeter scale configured to perform an impedance measurement (IPG).

The base 1010 may comprise a display 430, in particular an LED or e-ink screen or display, for displaying information to the user.

The base 1010 also includes a measuring plate 1020 suitable for receiving the user's feet. The measuring plate 1020 transmits the user's weight to the weight sensors.

The scale 1000 may further comprise a handle connected to the base 1010, not shown, suitable for gripping by at least one hand of the user.

Document FR3131524, for example, describes such a scale.

The physiological sensor 150 is arranged in the base 1010 and optionally in the handle.

Physiological sensor 150 can be a ballistograph or ballistocardiograph, BCG, in particular made up of weight sensors.

Alternatively or additionally, the physiological sensor 150 may be an impedance meter, IPG.

Alternatively or additionally, physiological sensor 150 can be an ECG sensor.

The ultrasonic transducer 180 is arranged in the base 1010 to be in contact with the skin of the user's feet.

### Other Embodiments

In an embodiment, the wearable device 100 is configured to be placed around the user's neck. In this case, the wearable device 100 takes the form of a necklace, for example.

In an embodiment, the wearable device 100 is configured to be placed at the user's head level. In this case, the wearable device 100 takes the form, for example, of headphones or a headband around the head.

In an embodiment, the portable device 100 is configured to be placed around the user's torso. In this case, the wearable device 100 takes the form of a bra or belt, for example.

In an embodiment, the wearable device 100 is configured to be placed around the user's leg. The wearable device 100 then takes the form, for example, of a band around the thigh.

The skilled person will understand that the wearable device 100 can be placed on any member of the user's body enabling both cardiac and ultrasound measurement.

### Activation Method

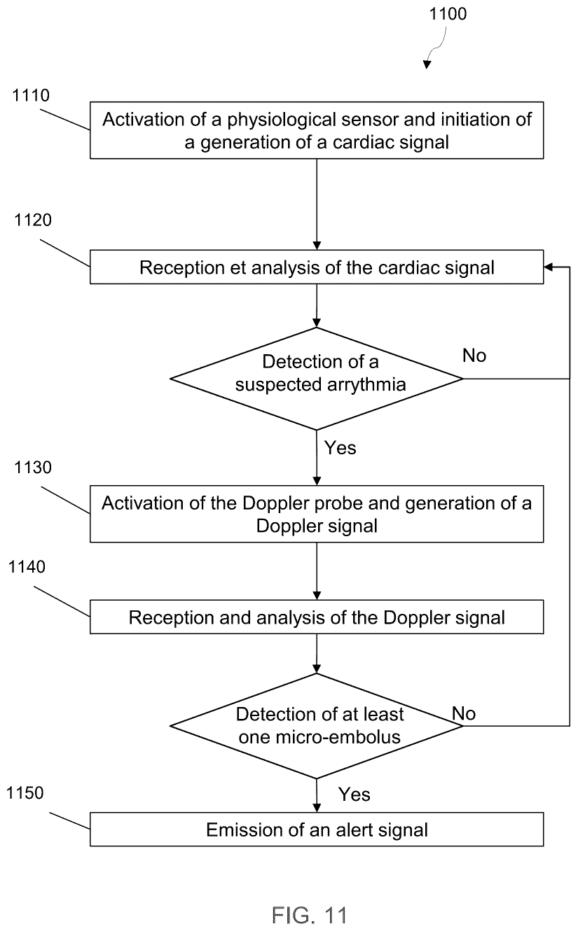

A method of activation 1100 of the ultrasonic transducer 180 implemented entirely by the control unit 140 will now be described, with reference to FIG. 11. As mentioned previously, in other embodiments, certain steps are implemented by the mobile terminal 460 and/or remote server 470.

The device 100 is positioned on a user's limb M, and in particular on the skin P of limb M.

Initially, ultrasonic transducer 180 is deactivated and therefore does not perform any measurements. In other words, ultrasonic transducer 180 does not emit ultrasonic signals in the direction of limb M.

In step 1110, the physiological sensor 150 is activated by the control unit 140. The control unit 140 controls the generation of a cardiac signal by the physiological sensor 150. The cardiac signal may be generated continuously or periodically. Physiological sensor 150 is typically activated continuously.

Then, in a step 1120, the control unit 140 receives the cardiac signal generated by the physiological sensor(s) 150 and analyzes the cardiac signal. The analysis is carried out continuously, and particularly in real time, for example with a sliding time window, as described above.

In response to the detection of a cardiac arrhythmia in the analysis of the cardiac signal in step 1120, the control unit 140 activates the ultrasonic transducer 180 in a step 1130 and initiates the generation of a Doppler signal by the ultrasonic transducer 180. Activation 1130 takes place in response to the detection of an arrhythmia, with no delay other than the inherent processing delays associated with the technique.

Then, in a step 1140, the control unit 140 receives the generated Doppler signal and analyzes it.

In response to the detection of a micro-embolus ME in the analysis of the Doppler signal in step 1140, the control unit 140 generates an alert signal in step 1150, in particular for the user and/or medical personnel.

The ultrasonic transducer 180 is then deactivated by the control unit 140, in particular to save the battery 120 of the device 100.

Method 1100 thus enables non-invasive, accurate detection of micro-emboli ME in users with atrial fibrillation. By integrating an ultrasound transducer 180 into a wearable device 100, such as a connected watch 500 or a blood pressure monitor 900, it is possible to detect micro-emboli ME following the detection of an arrhythmia, and thus enable rapid medical intervention, which is crucial for preventing damage caused by strokes.

In addition, activating the ultrasound transducer 180 only when an arrhythmia is detected enables optimized use of the battery 120 of the portable device 100, making long-term, continuous monitoring possible for the user.

In another embodiment, there is provided a non-transitory computer-readable medium storing instructions which, when executed by one or more processors of the control unit 140 of the device 100 as disclosed herein, cause the control unit 140 to implement a method comprising:
activating a physiological sensor 150 and initiating generation of a cardiac signal characteristic of a user's cardiac activity;
receiving and analyzing the cardiac signal to detect a suspected cardiac arrhythmia;
in response to detecting the suspected cardiac arrhythmia in the analysis of the cardiac signal, activating an ultrasonic transducer 180 to initiate generation of a Doppler signal characteristic of blood flow in a limb M of the user;
receiving and analyzing the Doppler signal to detect at least one micro-embolus ME; and
in response to detecting a flow of micro-emboli above a predetermined alert threshold, generating an alert signal intended for the user and/or a third party.

In an embodiment, the instructions, when executed, further cause the control unit 140 to: perform a classification of the cardiac signal using a machine-learning or deep-learning algorithm to assign the cardiac signal to one of a plurality of classes including at least “atrial fibrillation”, “other arrhythmia”, “sinus rhythm”, and “poor signal quality”; and determine the suspected cardiac arrhythmia based on a probability or confidence value output by the classification.

In an embodiment, the operations performed by the control unit 140 are implemented at least in part by software or firmware instructions executed by processor 412 and stored in memory 414. As described above, memory 414 may include volatile and/or non-volatile memory and can store one or more computer programs, algorithms or instruction sets that, when executed by processor 412, configure the control unit 140 to acquire and analyze the cardiac signal, to activate the ultrasonic transducer 180 in response to detection of a suspected arrhythmia, to acquire and analyze the Doppler signal and to generate one or more alert signals. The present description therefore also relates to a non-transitory computer-readable medium storing such instructions, the medium being readable by the processor 412 or by any equivalent processing circuitry.

In particular, an aspect of the invention relates to a computer program product comprising one or more sets of instructions which, when executed by one or more processors of the control unit 140, cause the control unit 140 to implement any of the methods described herein, including the activation method 1100 and the cardiac signal classification described above. The computer program product may be stored on a non-transitory computer-readable storage medium, such as a flash memory, ROM, EEPROM, optical disc, magnetic disc, solid-state memory or any other physical storage medium integrated in or operatively connected to the device 100. The computer program product is therefore not an abstract algorithm detached from any physical support, but is embodied in a physical medium and configured to control specific hardware components of the device 100.

When executed by processor 412, the instructions stored in memory 414 cause a functional cooperation between the software modules (e.g., PPG module 160, ECG module 170, oscillometric module 175 and Doppler module 195) and the physical sensors (physiological sensor(s) 150 and ultrasonic transducer 180). This cooperation results in a sequence of operations that begins with the physical acquisition of a cardiac signal on the user's body, continues with digital processing and classification of that signal to identify a suspected arrhythmia, and then triggers the physical emission and reception of ultrasonic waves in the user's limb to obtain a Doppler signal enabling detection of micro-emboli. The software therefore drives and coordinates concrete physical operations performed by the device, and the resulting outputs (e.g., embolic load estimation and alert signaling) are directly usable for medical diagnostic and therapeutic decision making.

In an embodiment, the information generated by execution of the instructions stored on the computer-readable medium is used as a diagnostic aid for a healthcare professional. For example, the device 100 may provide to a physician or telemedicine platform a diagnostic report comprising at least one indication of the presence or absence of atrial fibrillation, an estimate of the burden or frequency of micro-emboli detected during arrhythmic episodes, and one or more timestamps or summary indicators for high-risk events. These outputs are derived from real physiological measurements acquired on the user's body and processed by the claimed system; they are not arbitrary or user-entered data, but diagnostic indicators produced by the technical operation of the device. The computer-readable medium thus stores instructions that, when executed, contribute to solving a technical problem in the field of medical device technology, namely enabling reliable, energy-efficient, non-invasive detection of micro-emboli associated with arrhythmias in everyday conditions, using a compact device that can be worn or used by the patient.

The device and method described herein are not limited to a mere abstract manipulation of data, but are instead implemented by a concrete combination of hardware elements including at least one physiological sensor, an ultrasonic transducer and a control unit integrated into a wearable or non-invasive measuring device, such as a watch, a blood pressure monitor or a scale. The control unit is physically configured to acquire a cardiac signal from the physiological sensor, to process and analyze that signal in real time or near real time, to control activation of the ultrasonic transducer in response to the detected cardiac arrhythmia, and to receive and analyze Doppler signals representative of blood flow and micro-emboli in an artery of the limb. This specific interaction between hardware and software produces a tangible technical effect: a Doppler acquisition that is automatically synchronized with arrhythmic cardiac events, enabling non-invasive detection of micro-emboli temporally associated with the arrhythmia.

In particular, the method implemented by the control unit transforms raw physiological measurements (cardiac signal and Doppler signal) into clinically meaningful information regarding the presence of micro-emboli and the associated risk of stroke. The method therefore does not merely classify data in the abstract, but operates on signals physically measured on the user's body and directly controls a transducer that emits and receives ultrasonic waves in the user's limb. The resulting detection of micro-emboli, and the generation of an alert signal when an embolic burden above a threshold is identified, constitute concrete outputs that can be used by a healthcare professional to establish or refine a medical diagnosis, to initiate or adjust a therapy and to schedule further examinations. In other words, the system provides an automated diagnostic aid that is technically integrated within the measuring device and produces a real-world effect on patient management.

The invention further provides technical improvements over prior art systems that perform ECG or PPG monitoring in isolation, or that require separate, hospital-grade ultrasound equipment to investigate micro-embolic phenomena. In conventional approaches, long-term monitoring of atrial fibrillation and micro-emboli typically requires either short-duration Holter monitoring, which often misses transient arrhythmic episodes, or implantable cardiac monitors combined with intermittent transcranial Doppler examinations performed in specialized centers. These solutions are logistically complex, costly and not suitable for systematic, everyday screening of at-risk populations. By contrast, the present device integrates both cardiac sensing and Doppler ultrasound in a compact, wearable or home-usable form factor, enabling repeated or continuous monitoring in the user's usual environment, without the need for a dedicated ultrasound scanner or a specialized operator.

A further technical benefit resides in the specific control logic for activating the ultrasonic transducer only when a suspected arrhythmia has been detected in the cardiac signal, and substantially without delay. This conditional and tightly timed activation reduces power consumption of the ultrasonic transducer, thereby increasing battery life and enabling long-term continuous cardiac monitoring on a wearable device, while still ensuring that Doppler measurements are acquired during or immediately after arrhythmic events when micro-emboli are most likely to be present. This improves the signal-to-noise ratio and diagnostic relevance of the Doppler data, compared with systems that either acquire Doppler signals continuously (with prohibitive energy cost) or at arbitrary times unrelated to arrhythmia occurrence (with low likelihood of capturing embolic phenomena).

In addition, the use of advanced signal processing and, in some embodiments, deep learning algorithms in the control unit provides an improvement in the technical field of physiological signal processing. The algorithms are designed and trained to operate under the constraints of embedded hardware, to manage noise and motion artifacts inherent to wearable measurements, and to automatically differentiate various rhythms (such as sinus rhythm, atrial fibrillation and other arrhythmias) as well as to detect transient high-intensity Doppler events corresponding to micro-emboli. These algorithms are not generic classification routines, but are configured to drive and coordinate specific hardware components, including the physiological sensor and the ultrasonic transducer, in order to optimize the timing and quality of the measurements and to provide reliable, interpretable outputs for diagnostic use.

Accordingly, the claimed device and method solve a concrete technical problem: how to reliably and efficiently detect micro-emboli associated with arrhythmic cardiac events, in particular atrial fibrillation, using a compact, non-invasive device that can be worn or used by the patient in everyday life, while preserving battery autonomy and avoiding the need for heavy imaging infrastructure. The invention achieves this by a particular combination of sensors, control logic and signal-processing techniques that are functionally interlinked, and by generating a technically meaningful result (micro-embolus detection and alerting) that is directly usable in a medical diagnostic workflow. The invention is thus rooted in a specific practical implementation, produces a technical effect in the field of biomedical signal acquisition and processing, and goes beyond a mere abstract idea or mental process.

Expressions such as “comprise”, “include”, “incorporate”, “contain”, “is” and “have” are to be construed in a non-exclusive manner when interpreting the description and its associated claims, namely construed to allow for other items or components which are not explicitly defined also to be present. Reference to the singular is also to be construed in be a reference to the plural and vice versa.

The articles “a” and “an” may be employed in connection with various elements and components, processes or structures described herein. This is merely for convenience and to give a general sense of the compositions, processes or structures. Such a description includes “one or at least one” of the elements or components. Moreover, as used herein, the singular articles also include a description of a plurality of elements or components, unless it is apparent from a specific context that the plural is excluded.

As used herein in the specification and in the claims, the phrase “at least one”, in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

A person skilled in the art will readily appreciate that various features, elements, parameters disclosed in the description may be modified and that various embodiments disclosed may be combined without departing from the scope of the invention. For example, various aspects of the present disclosure may be used alone, in combination, or in a variety of arrangements not specifically described in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Having described above several aspects of at least one embodiment, it is to be appreciated various alterations, modifications, and improvements will readily occur to those skilled in the art. Such alterations, modifications, and improvements are intended to be aspects of this disclosure. Accordingly, the foregoing description and drawings are by way of example only.

1. A device configured to be positioned on a limb of a user, the device comprising:
a physiological sensor configured to generate a cardiac signal characteristic of a user's cardiac activity,
an ultrasound transducer configured to generate a Doppler signal characteristic of blood flow in the limb,
a control unit configured to:
receive a cardiac signal from the physiological sensor,
analyze the received cardiac signal, and
in response to a detection of a suspected cardiac arrhythmia in the cardiac signal analysis, activate the ultrasonic transducer to generate a Doppler signal.

2. The device according to claim 1, wherein the cardiac arrhythmia is atrial fibrillation.

3. The device according to claim 1, wherein the physiological sensor is an optical sensor configured to perform a continuous cardiac signal measurement and the control unit is configured to analyze the continuous cardiac signal.

4. The device according to claim 1, wherein the cardiac signal is analyzed using a sliding time window.

5. The device according to claim 1, wherein the physiological sensor is an ECG sensor, an oscillometric sensor, a ballistocardiograph, BCG, and/or an impedancemetric sensor, IPG.

6. The device according to claim 1, wherein the device is a wearable device.

7. The device according to claim 1, wherein the Doppler signal is generated concomitantly with the cardiac signal, or less than 10 seconds after the cardiac signal.

8. The device according to claim 1, wherein the ultrasonic transducer comprises at least a ultrasonic sensor, each ultrasonic sensor being configured to emit an ultrasonic signal and receive a signal reflected from the limb.

9. The device according to claim 8, wherein the ultrasonic transducer comprises several ultrasonic sensors.

10. The device according to claim 8, wherein the emitted ultrasonic signal is a constant-frequency pulsed signal.

11. The device according to claim 10, wherein the frequency of the emitted ultrasonic signal is between 8 MHz and 15 MHz.

12. The device according to claim 1, wherein the control unit is configured to receive the Doppler signal and configured to detect at least one micro-embolus in the artery by analysis of the Doppler signal.

13. The device according to claim 12, wherein the control unit is configured to emit an alert signal in the event of detection of a flow of micro-emboli above a predetermined alert threshold.

14. The device according to claim 1, wherein the device is a watch.

15. The device according to claim 1, wherein the device is a blood pressure monitor.

16. An activation method implemented by a control unit of a device according to claim 1, comprising:
activating the physiological sensor and initiating the generation of a cardiac signal;
receiving and analyzing the cardiac signal;
in response to a detection of a cardiac arrhythmia in the cardiac signal analysis, activating the ultrasonic transducer.

17. The activation method according to claim 16, further comprising initiating a generation of a Doppler signal by the ultrasonic transducer.

18. The activation method according to claim 17, further comprising receiving and analyzing the Doppler signal by the control unit.

19. The activation method according to claim 18, further comprising transmitting the Doppler signal to an external terminal or remote server for analysis.

20. The activation method according to claim 18, further comprising, in response to a detection of a micro-emboli, issuing an alert signal.
